(19) United States
(12) Patent Application Publication
(10) Pub. No.: US 2019/0257808 A1
(43) Pub. Date: Aug. 22, 2019

(54) Chemo-Metrical Prediction of Methane Index for the Natural Gas

(71) Applicant: The Regents of the University of California, Oakland, CA (US)

(72) Inventors: Chan Seung Park, Yorba Linda, CA (US); Partho Roy, Riverside, CA (US)

(73) Assignee: The Regents of University of California, Oaklad, CA (US)

(21) Appl. No.: 16/281,036

(22) Filed: Feb. 20, 2019

Related U.S. Application Data
(60) Provisional application No. 62/632,839, filed on Feb. 20, 2018.

(51) Int. Cl. G01N 33/22; F02D 41/14; G01N 33/28

(52) U.S. Cl. CPC G01N 33/225; F02D 41/1401; F02D 2041/1412; F02D 2200/0611; G01N 33/2835

(57) ABSTRACT

A new fuel quality sensor enables the use of RNG without removing CO₂. Efficiency and the economy of the process improves significantly if CO₂ separation cost can be avoided. Fuel quality information from the sensor makes it easy to adjust air/fuel ratio as well as efficient combustion inside a combustion engine or boiler.

### CROSS-REFERENCE TO RELATED APPLICATIONS

This Application claims the benefit of U.S. Provisional Patent Application No. 62/632,839, titled “Chemo-Metrical Prediction of Methane Index for the Natural Gas,” filed Feb. 20, 2018, the contents of which are incorporated herein by reference in their entirety.

### FEDERALLY SPONSORED RESEARCH AND DEVELOPMENT

This invention was made with Government support under Grant No. UCR-DOT-306 awarded by U.S. Department of Transportation. The government has certain rights in this invention.

### FIELD OF THE INVENTION

The invention relates to a chemo-metrical prediction of methane index for natural gas. Specifically, the invention relates to a method and system for predicting the Wobbe index and methane number of a renewable natural gas by the measurement of simple physical properties.

### BACKGROUND OF THE INVENTION

Renewable natural gas (RNG), i.e., natural gas produced from renewable feedstocks (e.g., landfill gas, anaerobic digestion gas, etc.) is an important alternative fuel that can contribute to achieving a number of goals set by local and federal government agencies relating to fossil fuel replacement and greenhouse gas (GHG) emissions reduction in the transportation sector. Most RNG production projects are small to medium scale by nature and comprehensive gas clean-up or upgrading to meet the fuel specifications of pipeline natural gas is often not feasible from a project economic perspective. This results in most RNG resources being wasted (e.g., flaring) or being left unused.

The RNG from a landfill site or anaerobic digester comes with a significant amount of CO₂; the CH₄ concentration varies from 35 to 70%, whereas CO₂ composition varies from 15 to 45%. RNG upgrading to pipeline natural gas requires removal of CO₂ from biogas as well as clean-up of impurities, drying, and compression. Among these series of processes, CO₂ removal is the most expensive, and it can cost more than $2/mcf.

The typical calorific value of RNG without CO₂ removal is around 50 to 60% of equal volume of fossil natural gas, and it varies significantly by project site and season.

Two of the most important parameters for fuel quality of natural gas are Wobbe Index (WI) and Methane Number (MN). WI is the ratio of the fuel calorific value to the square root of its specific gravity or relative density. WI is a critical factor in evaluating interchangeability of fuel gases such as natural gas and liquefied petroleum gas (LPG), and is used to compare the combustion energy output of different composition of fuel gases. Two fuels having identical WI have the identical energy output for a set of given operating condition. Therefore, WI is used in estimating the energy output in a wide variety of equipment and processes that involve natural gas (NG) combustion.

The MN of a gaseous fuel is defined as the methane composition (vol %) combined with hydrogen that makes the same knocking of the gas fuel under specified operating conditions in a knock testing engine. A different engine has a different MN range for suitable operation. MN is an important parameter in measuring engine performance, especially when the fuel is from renewable source with a high possibility of MN variation.

The WI is typically measured using bulky, complex and expensive analyzers. These devices measure the energy value of the fuel by direct calorimetry followed by a separate measurement of density by an optical method. The complex, destructive, and expensive nature of existing WI measurement systems prevent its use for on-site application. Traditionally MN is calculated from the gas composition. The gas composition can be measured by gas chromatography (GC) or a residual gas analyzer (RGA), which is time consuming and also requires a laboratory environment.

There is also a method and apparatus available for measuring the calorific value of a gas based on measuring the thermal conductivity value at two different temperatures and sound velocity values. An on-chip system that measures the energy content of fuel gases, such as natural gas, biogas and hydrogen, has also been proposed. A proposed thermal conductivity microsensor system uses a device that is able to detect the thermal conductivity of the gas mixture with insignificant errors, but the effect of temperature variation has not been studied. Another proposed an approach is to measure the MN and lower heating value of a gas online, which is valid for certain temperature and thermal conductivity of the gas. However, a variation of a wide range of temperature and pressure has not been studied. Studies for determining the gas mixture composition or energy content or thermal conductivity are available for either online or offline systems. Another proposed idea uses a thermal conductivity microsensor to measure the MN online, but no practical application or implementation of the proposed idea has been produced. Another study for designing and implementation of an analytical calculation model for determining the real-time composition of gas mixtures has been performed where the effects caused by the compressibility of the gas mixture and temperature are not taken into account in the model. The thermal conductivity (k) and sound velocity (v) through the gas is dependent on the temperature, pressure, density, and the composition of the gas mixture.

As discussed above, the previous research results show that the WI, MN, thermal conductivity, and sound velocity are dependent on the gas mixture. A limited scale of research also shows a possibility of estimating MN and gas composition using thermal conductivity and sound velocity instead of using the long and expensive process of using GC or RGA.

Given the shortcomings of the prior art, there is a need for a method and system for predicting the Wobbe Index and Methane Number of renewable natural gas by the measurement of simple physical properties. The present invention accomplishes this goal by using a predictive model that can accurately and efficiently estimate the WI, MN, and composition of the gas mixture based on only easily measurable physical properties, such as the thermal conductivity, sound velocity, temperature, and pressure of the gas to predict the quality and composition of gas in real-time.

### SUMMARY OF THE INVENTION

According to one aspect of a preferred embodiment, the WI is predicted by using a single thermal conductivity value.

According to yet another aspect of a preferred embodiment, a new fuel quality sensor enables the use of RNG without removing CO₂. Efficiency and the economy of the process improves significantly if CO₂ separation cost can be avoided. Fuel quality information from the sensor makes it easy to adjust air/fuel ratio as well as efficient combustion inside a combustion engine or boiler.

According to yet another aspect of a preferred embodiment, a method for fuel quality prediction enables the use of renewable natural gas (RNG) without removing CO₂. The calorific value of RNG without CO₂ removal is around 50 to 60% of fossil natural gas and varies significantly with RNG sources. Due to this fact, onsite measurement of the gas properties is preferable to ensure that expected quality is maintained, and better combustion efficiency is provided by adjusting the air/fuel ratio. Wobbe Index (WI) and Methane Number (MN) are the natural gas quality indicator and these parameters are dependent on the composition of gas mixture. This system and method builds a database for WI, MN, thermal conductivity and sound velocity through the gas mixture as a function of temperature, pressure and composition of the gas mixture. The system and method also develops a model for predicting the WI, MN and composition of the gas mixture. It takes temperature, pressure, thermal conductivity and sound velocity of the gas mixture and uses these variables to predict the WI, MN and composition. The prediction model can be based on MATLAB and may use the linear regression method for optimization.

In some embodiments, the system has been found to predict the WI and MN with an average prediction error of 1.386, and 0.559, respectively. In some embodiments, the system also predicts the composition of methane, ethane and CO₂ with an average error of 0.590, 0.279, and 0.509, respectively. The prediction model of the system, combined with a micro-sensor, has the potential to make the combustion of alternative fuel more efficient and ensure the quality of gaseous fuel supplied to an engine.

According to yet another aspect of a preferred embodiment, a database for WI, MN, thermal conductivity, sound velocity of a gas mixture is built; there is a prediction of WI and MN for a gaseous fuel mixture; a prediction of a compositions of the gaseous fuel mixture; and there is a prediction model coupled to a sensor that enables the combustion of gas efficiently.

### BRIEF DESCRIPTION OF THE DRAWINGS

These and other features, aspects and advantages of the present invention will become better understood with regard to the following description, appended claims, and accompanying figures where:

FIG. 1 is a high level schematic diagram that illustrates an apparatus for heating value calculation according to one embodiment;

FIG. 2 is a graph of the predicted Wobbe index verses the actual Wobbe index;

FIG. 3 is a graph of the predicted methane number verses the actual methane number;

FIG. 4 graph of the predicted CH₄ composition verses the actual CH₄ composition;

FIG. 5 is a graph of the predicted C₂H₆ composition verses the actual C₂H₆ composition; and

FIG. 6 is a graph of the predicted CO₂ composition verses the actual CO₂ composition.

FIG. 7 is an exemplary embodiment of a fuel system controller employing the current invention.

### DETAILED DESCRIPTION OF THE PREFERRED EMBODIMENTS

For illustrative purposes, the present invention is embodied in the apparatus and method generally shown and described herein with reference to FIG. 1 through FIG. 6. It will be appreciated that the apparatus may vary as to configuration and as to details of the parts, and that the method may vary as to the specific steps and sequence, without departing from the basic concepts as disclosed herein.

### Development of a Predictive Model

In order to develop a predictive model, a data set is developed. The system creates a data set that comprises attributes such as thermal conductivity, sound velocity, temperature, pressure, gas composition, WI, and MN. In this data set, the attributes, such as thermal conductivity, sound velocity, temperature, and pressure are physical properties that depend on the gas composition and are easily measured using widely available sensors or calculated through the gas mixture using software tools. The attributes, WI and MN also depend on the gas composition. Thus, in one embodiment, one method is to first measure the gas composition and calculate the WI and MN using the measured composition, which requires a long and expensive process. However, more preferably, the present system estimates WI, MN, and gas composition based on physical properties such as thermal conductivity, sound velocity, temperature, and pressure of the gas mixture in real-time. The main advantage of the system is that the physical properties can be easily and economically measured directly in the gas using sensors currently available in the market without analyzing the gas composition through the long and expensive process. A detailed process of preparing the data set and developing the predictive model to estimate the WI, MN, and gas composition will now be discussed.

### Creating a Data Set

To create a data set with the right attributes of gas composition, fossil, anaerobic digester gas, and landfill gas are mixed together to get the various combination of gas mixture containing components CH₄, CO₂ and C₂H₆ by 10% incremental from 0% to 100%. Table 1 illustrates the composition of the components in Fossil natural gas, anaerobic digester gas, and landfill gas.

TABLE 1

| Component of Gaseous | Fossil | Anaerobic | Landfill |
|---|---|---|---|
| Fuel Mixture | Gas | Digester Gas | Gas |
| Methane (mol %) | 97 | 68 | 60 |
| Carbon Dioxide (mol %) | 0 | 26 | 33 |
| Water (mol %) | 0 | 5 | 6.5 |
| Ethane (mol %) | 2 | 0 | 0 |
| Other (N2, O2) (mol %) | 1 | 1 | 0.5 |

Percent of Volumes of Components in Normalized Gases

| | | | |
|---|---|---|---|
| Methane (mol %) | 98 | 72.3 | 64.5 |
| Carbon Dioxide (mol %) | 0 | 27.7 | 35.5 |
| Ethane (mol %) | 2 | 0 | 0 |

For example, a mixture of 40% fossil, 30% anaerobic digester gas and 30% landfill gas contains 78.64% CH₄, 18.96% CO₂, and 2.4% C₂H₆. Using this method, by way of example, and not by way of limitation, sixty-six different combinations of the gas mixture is created for the gas composition. The combination of the gas mixture along with the composition of the components is shown in Table 2.

TABLE 2

| Gaseous Fuel Mixture Composition (%) | | | | | |
|---|---|---|---|---|---|
| Fossil | Anaerobic | Landfill | Composition (%) | | |
| Natural Gas | Disaster Gas | Gas | CH₄ | C₂H₆ | CO₂ |
| 100 | 0 | 0 | 94 | 6 | 0 |
| 90 | 10 | 0 | 91.83 | 5.4 | 2.77 |
| 80 | 0 | 20 | 88.1 | 4.8 | 7.1 |
| 70 | 20 | 10 | 86.71 | 4.2 | 9.09 |
| 60 | 30 | 10 | 84.54 | 3.6 | 11.86 |
| 50 | 30 | 20 | 81.59 | 3 | 15.41 |
| 40 | 40 | 20 | 79.42 | 2.4 | 18.18 |
| 30 | 20 | 50 | 74.91 | 1.8 | 23.29 |
| 20 | 50 | 30 | 74.3 | 1.2 | 24.5 |
| 10 | 60 | 30 | 72.13 | 0.6 | 27.27 |
| 10 | 20 | 70 | 69.01 | 0.6 | 30.39 |
| 0 | 100 | 0 | 72.3 | 0 | 27.7 |
| 0 | 0 | 100 | 64.5 | 0 | 35.5 |

In addition to the gas composition, pressure and temperature of the fuel are related to the thermal conductivity, sound velocity, WI, and MN. That indicates that thermal conductivity, sound velocity, WI, and MN may be determined by a function of temperature, pressure and composition of the gas mixture. Thus, combinations of temperature and pressure were created. For the combination of pressure, 500 psi intervals in the range between 500 psi and 3000 psi are used. For the combinations of temperature, 20° C. intervals in the range between −20° and 80° C. are used. From 6 different temperature intervals and 6 different pressure intervals, 36 possible combinations can be made. With these 36 combinations of temperature and pressure along, with 66 combinations of the gas mixture, the total of 2376 possible combinations were created.

The specific gas properties, such as thermal conductivity and sound velocity, are calculated. Thermal conductivity of the gaseous fuel mixture can be calculated by k=Σ xᵢkᵢ where kᵢ is the thermal conductivity of pure individual gas and xᵢ is the composition of the component in the mixture. The thermal conductivity data for each components at the specific T and P are taken as described in F. Uribe, E. Mason, J. Kestin, Thermal conductivity of nine polyatomic gases at low density, J. Phys. Chem. (1990), http://scitation.aip.org/content/aip/journal/jpcrd/19/5/10.1063/1.555864 (accessed Nov. 1, 2016), and B. A. Younglove, J. F. Ely, Thermophysical Properties of Fluids. II. Methane, Ethane, Propane, Isobutane, and Normal Butane, J. Phys. Chem. Ref. Data. 16 (1987) 577-798. doi:10.1063/1.555785. The thermal conductivity of the individual gas component in the mixture is calculated by equation 1 and equation 2. The equations are valid for methane when the reduced temperature above 1 and valid for other hydrocarbons at any temperature condition.

$$\begin{matrix}{k}_{i} = {10}^{- 7} {( 14.52 {T}_{r} - 5.14 )}^{2 / 3} \frac{{C}_{p}}{λ} & [ equation 1 ] \\ λ = T {c}^{1 / 6} {{M}^{1 / 2} ( \frac{101.325}{{p}_{c}} )}^{2 / 3} & [ equation 2 ]\end{matrix}$$

where kᵢ=vapor thermal conductivity of pure components, W/m K; Tr=reduced temperature, T/T_{c}; T=temperature, K; T_{c}=critical temperature, K; Cₚ=heat capacity at constant pressure, J/kmol K; M=molecular weight and p_{c}=critical pressure, kPa.

Sound velocity of the mixed gas is calculated by the following equation: Sound velocity,

$$c = \sqrt{\frac{ ZRT}{{M}_{g}}} \qquad [ equation 3 ]$$

where

$$= \frac{{C}_{p}}{{C}_{v}} ;$$

Z=compressibility factor; Constant pressure specific heat, Cₚ=Σ/(xᵢCₚᵢ(Tᵢ)) [J/mol·K].

Temperature based constant pressure heat capacity for each component is calculated for ideal gas condition.

Constant volume specific heat, C_{y}=Cₚ−R[J/mol·K]

where the compressibility factor is calculated as a function of pseudo reduced pressure and temperature. Pseudo reduced pressure and temperature are calculated by using Suttons gas gravity method:

$$\begin{matrix}{p}_{pc} = 7.56 .8 - 131.07 - 3.6 {2}^{} & [ equation 4 ] \\ {T}_{pc} = 169.2 + 349.5 - 74.0 {2}^{} & [ equation 5 ] \\ {p}_{pr} = \frac{p}{{p}_{pc}} & [ equation 6 ] \\ {T}_{pr} = \frac{T}{{T}_{pc}} & [ equation 7 ]\end{matrix}$$

where relative density/Specific gravity

$$( ) = \frac{Molecular weight of the gas ( {M}_{g} )}{Molecular weight of air ( {M}_{a} )}$$

Molecular weight of the gas mixture, M_{g}=Σ/Mᵢxᵢ where Mᵢ is the molecular weight of the component and xᵢ is the composition of the component.

WI for the gas mixture is calculated by the following equation:

$$WI = \frac{{H}_{c}}{\sqrt{}} \qquad [ equation 8 ]$$

where H_{c} is the heating value of the gas mixture at the specific temperature and pressure and [symbol] _{g} is the relative density/specific gravity of the mixed gas.

An Aspen Plus process simulator is used for calculating the heating value of combustible components. A diagram of the apparatus developed by Aspen Plus is shown in FIG. 1. In the model, the combustible components CH₄ and C₂H₆ are burned in presence of stoichiometric air ratio and the product leaves the reactor at the reaction temperature and pressure (at which temperature and pressure Wobbe index is calculated). CO₂ also contributes to increase/decrease the Wobbe index of the mixed gas by changing the volume not by changing the total heat content.

The methane number of gas mixture is obtained from Cummins Westport's (CWI) web site, http://www.cumminswestport.com/fuel-quality-calculator (accessed Jul. 27, 2017). CWI uses the SAE based methane number calculation (SAE 922359 equation 4). In 2015, CWI's fuel quality calculator switched to a Cummins Proprietary methane number calculation. The Cummins Proprietary calculation provides a more accurate representation of the true MN of the fuel. Table 3 illustrates a portion of the data set including all the attributes for physical properties, WI, MN, and gas composition.

TABLE 3

| | | Composition | | | Thermal | Sound | Wobbe | Methane |
|---|---|---|---|---|---|---|---|---|
| Temperature, | Pressure, | CH₄ | C₂H₆ | CO₂ | Conductivity, | Velocity, | Index | Number |
| T [K] | P [kPa] | % | % | % | k × 10³ [W/m · K] | v [m/s] | [MJ/Nm³] | [MN] |
| 253.15 | 3447 | 94 | 6 | 0 | 27.36 | 350.86 | 54.9 | 84.5 |
| 253.15 | 3447 | 91.83 | 5.4 | 2.77 | 27.02 | 343.9 | 52.16 | 88.8 |
| 253.15 | 3447 | 89.66 | 4.8 | 5.54 | 26.68 | 336.48 | 49.53 | 93.2 |
| 253.15 | 3447 | 87.49 | 4.2 | 8.31 | 26.33 | 330.29 | 47.03 | 97.6 |
| 253.15 | 3447 | 85.32 | 3.6 | 11.08 | 25.99 | 324.43 | 44.62 | 102 |
| 253.15 | 3447 | 83.15 | 3 | 13.85 | 25.64 | 318.88 | 42.32 | 106.4 |
| 253.15 | 3447 | 80.98 | 2.4 | 16.62 | 25.3 | 313.6 | 40.1 | 110.7 |
| 253.15 | 3447 | 78.81 | 1.8 | 19.39 | 24.95 | 307.78 | 37.97 | 115 |
| 253.15 | 3447 | 76.64 | 1.2 | 22.16 | 24.61 | 303 | 35.91 | 119.2 |
| 253.15 | 3447 | 74.47 | 0.6 | 24.93 | 24.26 | 298.44 | 33.92 | 123.3 |
| 253.15 | 3447 | 72.3 | 0 | 27.7 | 23.92 | 294.08 | 32 | 127.3 |
| 253.15 | 3447 | 71.52 | 0 | 28.48 | 23.8 | 292.74 | 31.51 | 128.1 |
| 253.15 | 3447 | 73.69 | 0.6 | 25.71 | 24.14 | 297.04 | 33.41 | 124.2 |
| 253.15 | 3447 | 70.74 | 0 | 29.26 | 23.68 | 291.42 | 31.03 | 129 |
| 253.15 | 3447 | 75.86 | 1.2 | 22.94 | 24.49 | 301.54 | 35.38 | 120.1 |
| 253.15 | 3447 | 72.91 | 0.6 | 26.49 | 24.03 | 295.66 | 32.91 | 125 |
| 253.15 | 3447 | 69.96 | 0 | 30.04 | 23.56 | 290.11 | 30.55 | 129.8 |
| 253.15 | 3447 | 78.03 | 1.8 | 20.17 | 24.83 | 306.24 | 37.42 | 115.9 |
| 253.15 | 3447 | 75.08 | 1.2 | 23.72 | 24.37 | 300.09 | 34.86 | 121 |
| 253.15 | 3447 | 72.13 | 0.6 | 27.27 | 23.91 | 294.3 | 32.41 | 125.9 |
| 253.15 | 3447 | 69.18 | 0 | 30.82 | 23.45 | 288.83 | 30.07 | 130.7 |
| 253.15 | 3447 | 80.2 | 2.4 | 17.4 | 25.18 | 311.98 | 39.53 | 111.6 |

### Constructing a Predictive Model

In a statistical modelling, regression analysis is commonly used for estimating the relationships among variables in a data set. Among many techniques for analysing a data set, a multiple regression approach is used to build a predictive model that estimates the WI. MN, and gas composition. Multiple regression allows for multiple dependent and independent variables, often called predictors, as well as multiple coefficients, β.

The general multiple regression equation for a dependent variable, yᵢ is

yᵢ=β₀+β₁x₁ᵢ+ . . . +βₘxₘᵢ+εᵢ for i=1 . . . n [equation 9]

where xₘᵢ represents independent variables; β represents the coefficients that are normally unknown but to be found; ε represents the error; n represents the number of dependent values (that is equal to the samples); m represents the number of independent variables. Based on this, a multiple regression model is typically presented in the following form:

Y=Xβ+ε [equation 10]

where Y is a vector representing values of dependant variables; X is a matrix representing values of independent variables. In most regression analysis, the value of ε is assumed to be randomly distributed. A regression model in general relates or approximates Y to a function of X and β, that is, Y=f(X, β). In order to find a functional relational relationship between X and Y, an appropriate number of samples (or measurements) may be used as shown below, where n is number of samples and m is the number of variables. The data collection process for the sample data set used in this modelling is described above.

$$Y = [ \begin{matrix}{y}_{1} \\ {y}_{2} \\ ⋮ \\ {y}_{n}\end{matrix} ] , X = [ \begin{matrix}1 & {x}_{11} & {x}_{21} & ⋯ & {x}_{k 1} \\ 1 & {x}_{12} & {x}_{22} & ⋯ & {x}_{k 2} \\ ⋮ & ⋮ & ⋮ & ⋱ & ⋮ \\ 1 & {x}_{1 n} & {x}_{2 n} & ⋯ & {x}_{kn}\end{matrix} ] , β = [ \begin{matrix}{β}_{1} \\ {β}_{2} \\ ⋮ \\ {β}_{2}\end{matrix} ] , ɛ = [ \begin{matrix}{ɛ}_{1} \\ {ɛ}_{2} \\ ⋮ \\ {ɛ}_{2}\end{matrix} ]$$

Y represents a vector consisting of WI, MN, and gas composition of all three components as dependent variables. X is a matrix representing the values of 2376 samples for all the physical properties as independent variables. The goal is to find a vector \tilde{β} representing coefficients for the model that minimizes the square sum of the error, that is \tilde{β}=argmin_{β}Σᵢ₌₁ⁿ(yᵢ−β^{T}xᵢ)². The coefficients (β-vector) are calculated using the Least Squares Equation:

\tilde{β}=(X^{T}X)⁻¹X^{T}Y [equation 11 ]

With the calculated coefficients \tilde{β}, the estimated Y, Ŷ is calculated by Ŷ=X\tilde{β}.

Further, the average error of the model is calculated by the root mean square (rms) between the actual value in the data set and the predicted value by the model as shown by the formula below:

$${error}_{r m s} = \sqrt{\frac{{Σ error}^{2}}{2376}} \qquad [ equation 12 ]$$

### Results from the System

The system to predict the WI, MN, and gas composition from the physical properties is developed based on multiple regression using MATLAB.

### Wobbe Index Prediction Model

The model is created base on four different variables: constant (1),

$$\frac{1}{temperature ( T )} ( {x}_{1} ) , \\ \frac{1}{pressure ( P )} ( {x}_{2} ) , \\ \frac{1}{thermal conductivity ( k )} ( {x}_{3} ) , and$$

$$\frac{1}{sound velocity ( v )} ( {x}_{4} ) .$$

Y is the matrix representing the WIs for all the samples in the data set. The coefficients for the model are:

$$β = [ \begin{matrix}70.76 \\ - 0.55 \\ 0.000225 \\ 3635.79 \\ 0.05\end{matrix} ] .$$

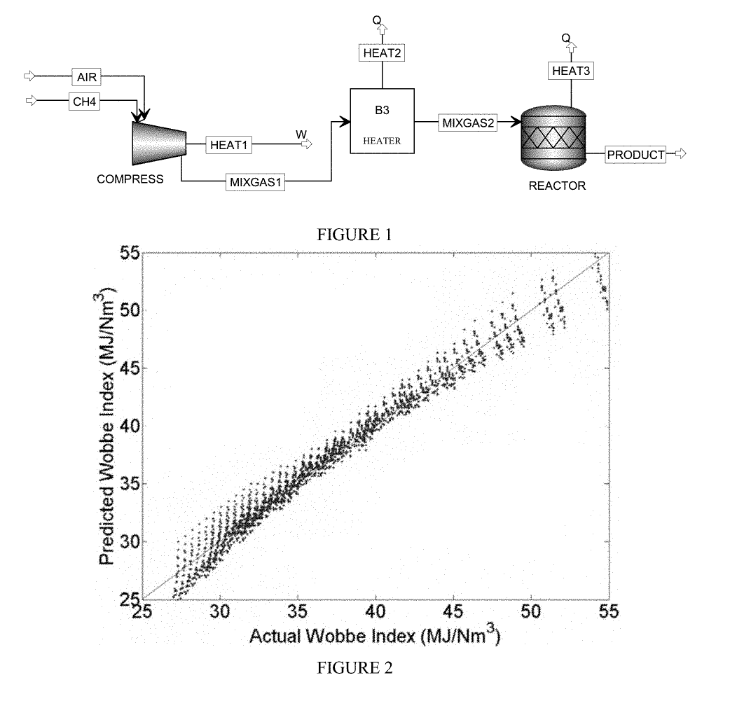

FIG. 2 shows the comparison of actual value and predicted value of WI. The trend line is desirable. While the accuracy of the predicted WIs in the range of 30-40 is very high, it is dropped slightly outside the range. The average error, error_{rms is} 1.02 and the average percentage of error was 2.76%. Table 4 also shows the detailed regression statistics for the WI prediction model. These statistics strongly support the accuracy of the model for WI.

TABLE 4

| R Square | 0.98 | | |
|---|---|---|---|
| Standard Error | 0.94 | | |
| | Standard Error | t Stat | P-value |
| Intercept (constant) | 0.48 | 238.35 | 0.00 |
| Variable 1 | 0.00 | −171.64 | 0.00 |
| (Temperature, T) | | | |
| Variable 2 | 0.00 | −22.29 | 0.00 |
| (Pressure, P) | | | |
| Variable 3 | 37.38 | 112.05 | 0.00 |
| (Thermal Conductivity, k) | | | |
| Variable 4 | 0.00 | 24.07 | 0.00 |
| (Sound Velocity, v) | | | |

The model for MN prediction is developed using four different variables: constant (1),

$$\frac{1}{temperature ( T )} ( {x}_{1} ) , \\ \frac{1}{pressure ( P )} ( {x}_{2} ) , \\ \frac{1}{thermal conductivity ( k )} ( {x}_{3} ) , and$$

$$\frac{1}{sound velocity ( v )} ( {x}_{4} ) .$$

Y is the matrix representing the methane number for all the samples. The coefficients for the model are:

$$β = [ \begin{matrix}51.71 \\ 1.06 \\ 0.000402 \\ - 7240.22 \\ - 0.10\end{matrix} ] .$$

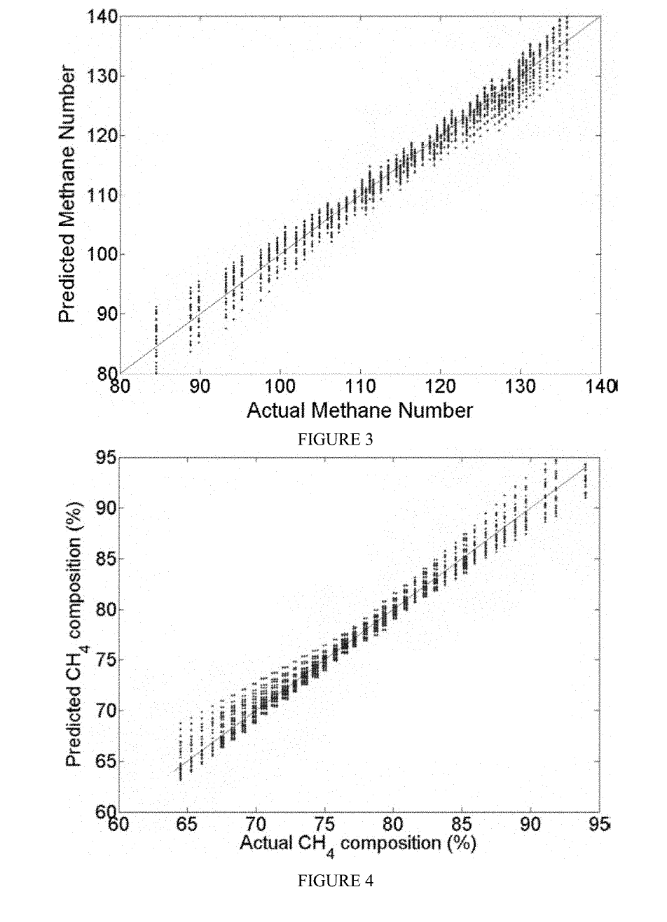

FIG. 3 shows the accuracy of the predicted values of methane number. As illustrated in the figure, the accuracy of the predicted MNs is much higher than the predicted WIs. The average error, errorᵣₘₛ was 1.92 and the average percentage of error was 1.65%. Table 5 shows the detailed regression statistics for the MN prediction model.

TABLE 5

| R Square | 0.98 | | |
|---|---|---|---|
| Standard Error | 1.92 | | |
| | Standard Error | t Stat | P-value |
| Intercept (constant) | 0.98 | 52.67 | 0.00 |
| Variable 1 | 0.01 | −147.54 | 0.00 |
| (Temperature, T) | | | |
| Variable 2 | 0.00 | −22.66 | 0.00 |
| (Pressure, P) | | | |
| Variable 3 | 76.20 | 95.01 | 0.00 |
| (Thermal Conductivity, k) | | | |
| Variable 4 | 0.00 | 24.47 | 0.00 |
| (Sound Velocity, v) | | | |

### Gas Composition Prediction Model

The model for the gas composition prediction is developed using four different variables: constant (1),

$$\frac{1}{temperature ( T )} ( {x}_{1} ) , \\ \frac{1}{pressure ( P )} ( {x}_{2} ) , \\ \frac{1}{thermal conductivity ( k )} ( {x}_{3} ) , and$$

$$\frac{1}{sound velocity ( v )} ( {x}_{4} ) .$$

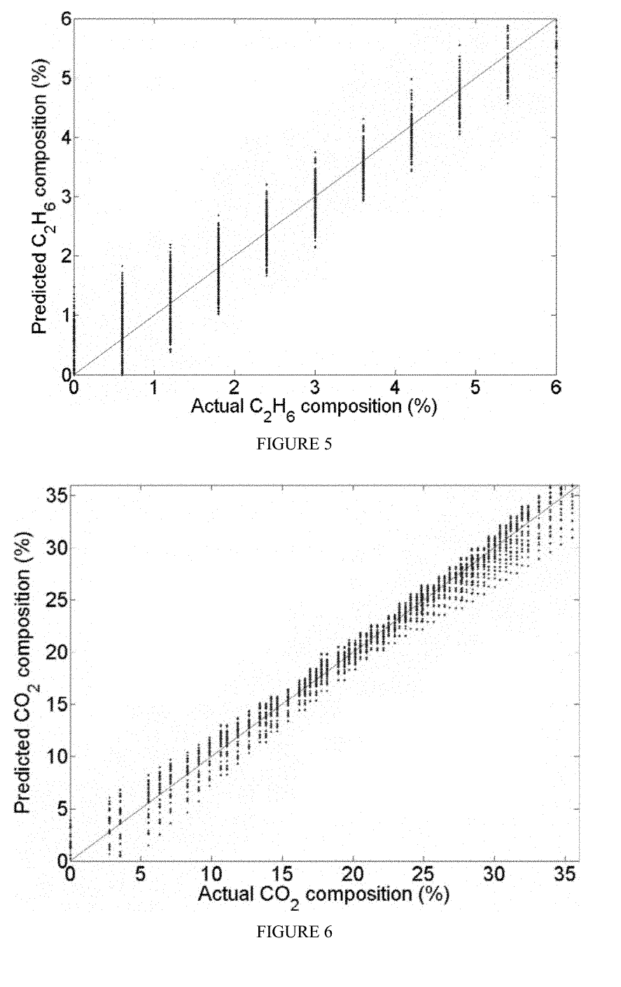

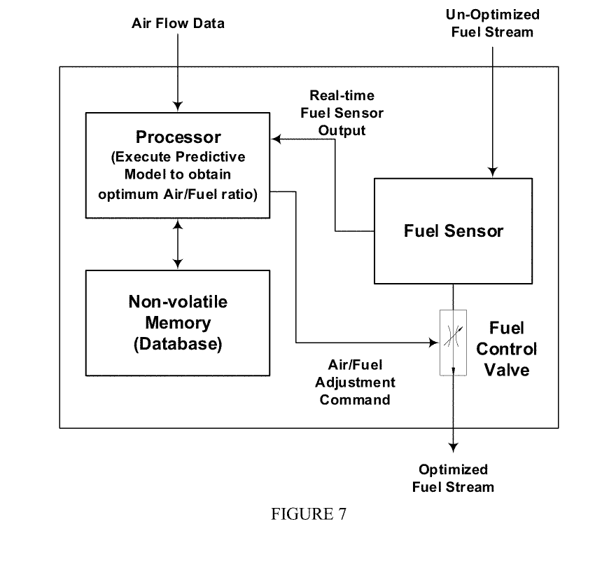

Table 6 shows the detailed regression statistics for the Gas Composition prediction model. The predicted data vs. the actual data plot for CH₄, C₂H₆, and CO₂ are presented in FIG. 4, FIG. 5, and FIG. 6, respectively.

TABLE 6

| | CH₄ | C₂H₆ | CO₂ | | | | | | |
|---|---|---|---|---|---|---|---|---|---|
| R Square | 0.98 | 0.93 | 0.98 | | | | | | |
| Standard Error | 0.94 | 0.43 | 1.16 | | | | | | |
| | Standard Error | | | t Stat | | | P-value | | |
| | CH₄ | C₂H₆ | CO₂ | CH₄ | C₂H₆ | CO₂ | CH₄ | C₂H₆ | CO₂ |
| Intercept (constant) | 0.48 | 0.22 | 0.59 | 238.35 | 43.39 | 40.99 | 0.00 | 0.00 | 0.00 |
| Variable 1 (Temperature, T) | 0.00 | 0.00 | 0.00 | −171.64 | −79.11 | 168.40 | 0.00 | 0.00 | 0.00 |
| Variable 2 (Pressure, P) | 0.00 | 0.00 | 0.00 | −22.29 | 14.13 | 23.30 | 0.00 | 0.00 | 0.00 |
| Variable 3 (Thermal Cond., k) | 37.38 | 17.16 | 46.15 | 112.05 | 50.21 | −109.40 | 0.00 | 0.00 | 0.00 |
| Variable 4 (Sound Velocity, v) | 0.00 | 0.00 | 0.00 | 24.07 | 15.26 | −25.17 | 0.00 | 0.00 | 0.00 |

The predicted methane composition shown in FIG. 4 shows negligible error in the range where methane composition is in the range of 70 to 85%. The predicted data out of the range has the largest fluctuation. The prediction of methane composition is very closely connected with the WI prediction since WI is mostly contributed by combustible component methane and ethane. Thus, as the ethane concentration is very small, the predicted methane composition and WI in the range is accurate. The errormᵣₘₛ for methane composition prediction model is 0.94 and the average percentage of error was 1.22%.

FIG. 5 shows the predicted and actual ethane composition. As illustrated in the figure, with limited number of data points, the prediction of ethane composition is relatively inaccurate. The errorᵣₘₛ for this prediction was 0.43 and the average percentage of error was 5.51%.

FIG. 6 shows the predicted CO₂ composition vs actual CO₂ composition. Particularly, the accuracy of the prediction is very high where the CO₂ composition is above 10%. The errorᵣₘₛ for CO₂ composition prediction model was 1.16 and the average percentage of error was 21.59%.

### Conclusion

The development in worldwide alternative fuel use is significantly dependent on the efficient combustion compatibility of the fuel in engine. WI and MN are the fuel quality indicator and the proper information on WI can make the combustion process efficient for the engine. In this research, in order to develop a predictive model that estimate WI, MN, and the composition of the gas, a data set is created that includes temperature, pressure, thermal conductivity, sound velocity, WI, MN, and composition of the gas mixture from fossil natural gas, anaerobic digester gas and landfill gas. The model takes thermal conductivity, sound velocity, temperature and pressure and predict WI, MN, and the gas composition. A database is developed for thermal conductivity, sound velocity, WI and MN of the gas mixture dependent on the temperature, pressure, and composition of the gas mixture. A model for prediction of WI, MN and composition of the components of fossil natural gas blended with anaerobic digester gas and landfill gas is developed.

The system can predict a WI and an MN of a gas mixture and also efficiently predicts the composition of methane, ethane and CO₂ in the gas mixture. The prediction model, once coupled with a gas sensor, has the potential to make the combustion of alternative fuel more efficient.

One possible embodiment can be an engine controller that adjusts the air/fuel ratio for the specific input gas for optimum efficiency. For some controllers, the adjustment can be automated and in real-time. An onboard engine controller can comprise: a non-resident memory containing a database of Wobbe index values, methane number values, thermal conductivity values, and sound velocities of a gas mixture; a gas sensor to provide real time parameters of the fuel input stream; and a processor to run a predictive model coupled to the database and the sensor to determine the real-time parameters of the fuel input stream, process air flow input data, and provide real-time air/fuel ratio adjustment commands based on the predictive model; and a fuel control value to adjust the air/fuel ratio based on the adjustment command; whereby the air/fuel ratio is optimized for an engine based on the specific fuel provided. In some controller embodiments, the processor can further host a data set builder for the predictive model to build the database of Wobbe index values, methane number values, thermal conductivity values, and sound velocities of a gas mixture. In some controllers, the processor executes a linear regression predictive model.

The various embodiments described above are provided by way of illustration only and should not be construed to limit the invention. Those skilled in the art will readily recognize various modifications and changes that may be made to the claimed invention without following the example embodiments and applications illustrated and described herein, and without departing from the true spirit and scope of the claimed invention, which is set forth in the following claims.

What is claimed is:

1. A fuel quality sensor, comprising:
a database of Wobbe index values, methane number values, thermal conductivity values, and sound velocities of a gas mixture; and
a predictive model coupled to the database and a sensor to determine parameters of renewable nature gas and to adjust an air/fuel ratio for an engine based on the predictive model.

2. The system of claim 1, further comprising a data set builder for the predictive model that builds the database.

3. The system of claim 1, wherein the predictive model comprises a linear regression predictive model.

4. The system of claim 1, wherein the predictive model is to adjust the air/fuel ratio in real time during operation of the engine.

5. A method for sensing fuel quality, the method comprising:
building a database of Wobbe index values, methane number values, thermal conductivity values, and sound velocities of a gas mixture; and
applying a predictive model coupled to the database and a sensor to determine parameters of renewable nature gas and to adjust an air/fuel ratio for an engine based on the predictive model.

6. The method of claim 5, wherein the step of building the database comprises using a data set builder for the predictive model.

7. The method of claim 6, wherein the predictive model comprises a linear regression predictive model.

8. The method of claim 5, wherein the predictive model is to adjust the air/fuel ratio in real time during operation of the engine.

9. An onboard engine controller comprising:
a non-resident memory containing a database of Wobbe index values, methane number values, thermal conductivity values, and sound velocities of a gas mixture;
a gas sensor to provide real time parameters of the fuel input stream;
a processor to run a predictive model coupled to the database and the sensor to determine the real-time parameters of the fuel input stream, process air flow data, and provide real-time air/fuel ratio adjustment commands based on the predictive model;
a fuel control value to adjust the air/fuel ratio based on the adjustment command;
whereby the air/fuel ratio is optimized for an engine based on the specific fuel provided.

10. The controller of claim 9, where the processor further hosts a data set builder for the predictive model that builds the database.

11. The controller of claim 9, where the processor executes a linear regression predictive model.
